(19) United States
(12) Patent Application Publication
(10) Pub. No.: US 2010/0305400 A1
(43) Pub. Date: Dec. 2, 2010

(54) MEDICAL DEVICE AND ENDOSCOPE

(76) Inventors: Fumiyuki Onoda, Tama-shi (JP); Masanori Hamazaki, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 12/826,112

(22) Filed: Jun. 29, 2010

Related U.S. Application Data
(63) Continuation of application No. PCT/JP2008/050701, filed on Jan. 21, 2008.

(51) Int. Cl. A61B 1/00

(52) U.S. Cl. 600/104

(57) ABSTRACT

A medical device includes a first medical device, a second medical device, an engagement mechanism, a connection release mechanism and a detachment mechanism. The second medical device is attachable to/detachable from the first medical device. The engagement mechanism includes an engaging section provided on the first medical device, and an engaged section provided on the second medical device and engaged in the engaging section. The connection release mechanism includes an external-force input section applied with an external force for releasing connection between the first and second medical devices. The detachment mechanism includes a detachment section where the engaging section or the engaged section is irreversibly detached from the second medical device when the external force is applied to the external-force input section.

### CROSS-REFERENCE TO RELATED APPLICATIONS

This is a Continuation Application of PCT Application No. PCT/JP2008/050701, filed Jan. 21, 2008, which was published under PCT Article 21(2) in Japanese.

### BACKGROUND OF THE INVENTION

1. Field of the Invention

The present invention relates to a medical device and an endoscope of a disposable type.

2. Description of the Related Art

For example, in an endoscope disclosed in Jpn. Pat. Appln. KOKAI Publication No. 10-258028, a piping unit which is disposable can be attached to/detached from an insertion section and an operation section which are reused. Further, when the disposable piping unit is detached from the operation section to be reused, a front wall of an engagement groove of the piping unit is broken by an annular convex on a suction pipe in a side of the operation section. The endoscope thus prevents reuse of the disposable piping unit.

### BRIEF SUMMARY OF THE INVENTION

A medical device includes a first medical device, a second medical device, an engagement mechanism, a connection release mechanism and a detachment mechanism. The second medical device is attachable to/detachable from the first medical device. The engagement mechanism includes an engaging section provided on the first medical device, and an engaged section provided on the second medical device and engaged in the engaging section. The connection release mechanism includes an external-force input section applied with an external force for releasing connection between the first and second medical devices. The detachment mechanism includes a detachment section where the engaging section or the engaged section is irreversibly detached from the second medical device when the external force is applied to the external-force input section.

### BRIEF DESCRIPTION OF THE SEVERAL VIEWS OF THE DRAWING

FIG. 1A is a schematic view illustrating an endoscope system according to the first to fourth embodiments;

FIG. 1B is a schematic view illustrating a configuration of a distal end surface of a distal end of an insertion section of the endoscope system according to the first to fourth embodiments;

FIG. 2A is a schematic view illustrating a state in which a connector of the endoscope and a connector receiving section of a light source device are connected to each other, in the endoscope system according to the first embodiment;

FIG. 2B is a schematic perspective view illustrating the connector of the endoscope in the endoscope system according to the first embodiment;

FIG. 3 is a schematic longitudinal cross-sectional view illustrating a structure of a flexible pipe section of the insertion section of the endoscope in the endoscope system according to the first embodiment;

FIG. 4A is a schematic perspective view illustrating a claw member provided on the connector of the endoscope, a connector fixed to the claw member, a universal cable, an operation section, and wires provided for the insertion section, in the endoscope system according to the first embodiment;

FIG. 4B is a schematic perspective view illustrating a claw member provided on the connector of the endoscope, the connector fixed to the claw member, the universal cable, the operation section, and the wires provided for the insertion section, in the endoscope system according to the first embodiment;

FIG. 5 is a schematic perspective view illustrating a state in which the wires fixed to the claw member are inserted through wire insertion holes in a tube which is used for the flexible pipe section or the universal cable of the insertion section of the endoscope, in the endoscope system according to the first embodiment;

FIG. 6 is a schematic view illustrating a state in which a connector of the endoscope and a connector receiving section of a light source device are connected to each other, in the endoscope system according to the second embodiment;

FIG. 7A is a schematic cross-sectional view illustrating a flexible pipe section of the insertion section of the endoscope in the endoscope system according to the third embodiment;

FIG. 7B is a schematic view illustrating a state in which an operation-section-side operation wire and a distal-end-side operation wire of the insertion section among operation wires are connected, inside the insertion section or an operation section of the endoscope in the endoscope system according to the third embodiment;

FIG. 8A is a schematic view illustrating a state in which a connector of the endoscope and a connector receiving section of a light source device are connected to each other, in the endoscope system according to the fourth embodiment;

FIG. 8B is a schematic view illustrating a state immediately before the connector of the endoscope and the connector receiving section of the light source device are connected to each other, in the endoscope system according to the fourth embodiment; and

FIG. 8C is a schematic view illustrating a state immediately before the connector of the endoscope and the connector receiving section of the light source device are connected to each other, in the endoscope system and also illustrating a modification to the connector of the endoscope illustrated in FIG. 8B according to the fourth embodiment.

### DETAILED DESCRIPTION OF THE INVENTION

### First Embodiment

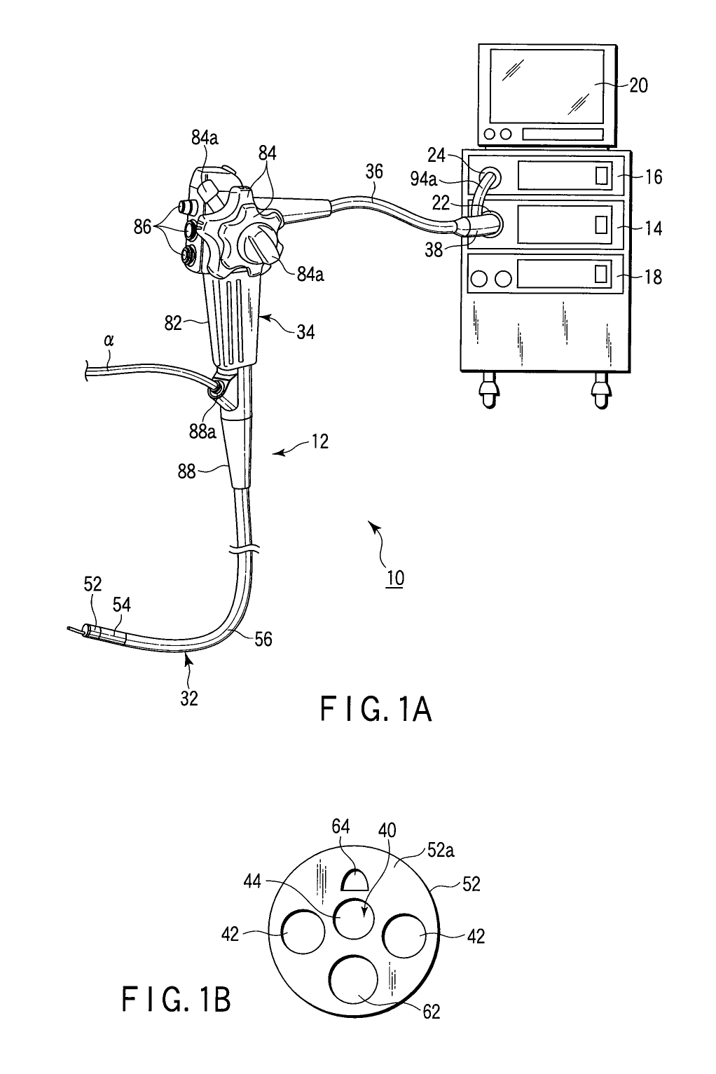

The first embodiment will be described first with reference to FIG. 1A to FIG. 5.

As illustrated in FIG. 1A, an endoscope system (medical device) 10 according to this embodiment includes an endoscope (first medical device) 12 of a disposable type, and a light source device (second medical device) 14, a processor 16, a solenoid valve 18, and a monitor 20, each of which is reusable.

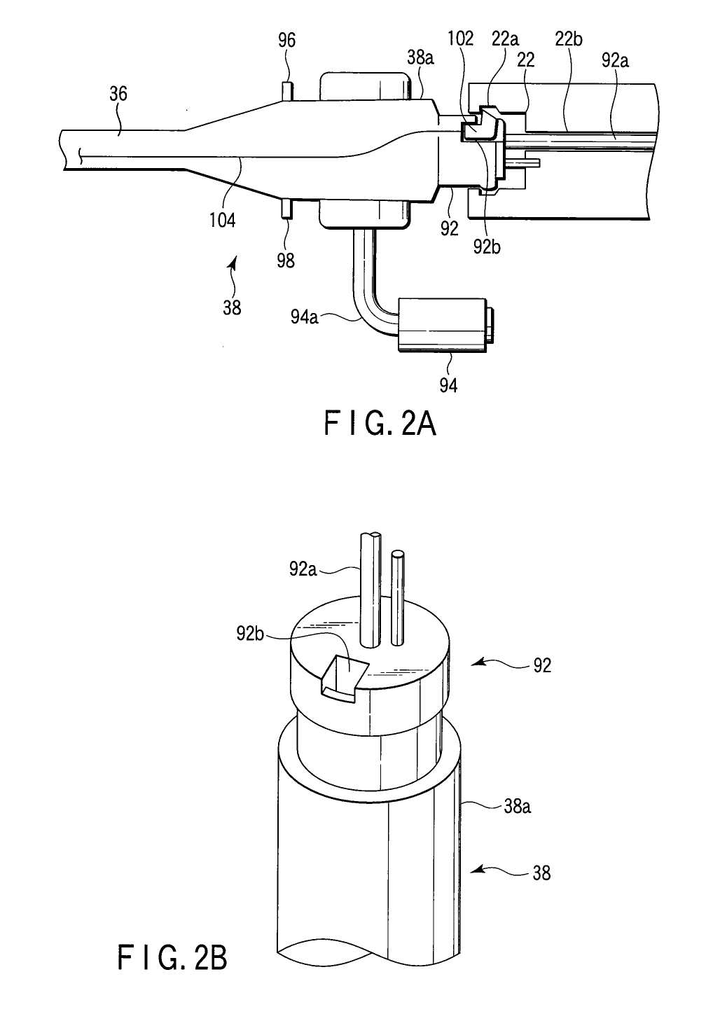

The light source device 14 supplies illumination light to a light guide bundle of an illumination optical system described later to illuminate an object. Further, a light-source-device-connector receiving section 22 where a light guide connector 92 of a connector 38 described later of the endoscope (endoscope main body) 12 is provided is formed on the light source device 14. As illustrated in FIG. 2A, a concave (engaged section, engagement means) 22a to be engaged with a claw member 102 described later is formed on the light-source-device-connector receiving section 22. Also, a rod guide 22b where a light guide rod 92a, described later, of the light guide connector 92 is provided is formed on the light-source-device-connector receiving section 22. A light source (unillustrated) is provided behind the rod guide 22b.

The endoscope 12, light source device 14, solenoid valve 18, and monitor 20 are detachably connected to the processor 16 illustrated in FIG. 1A, which controls these components in desired states. Specifically, a processor-connector receiving section 24 where an electric connector 94 of the connector 38, described later, of the endoscope 12 is provided is formed for the processor 16. Further, the processor 16 receives operations from a switch 86 in an operation section 34, described later, of the endoscope 12, and automatically controls the light source device 14, solenoid valve 18, and monitor 20.

Based on the operations on the switch 86 of the operation section 34, described later, of the endoscope 12, the solenoid valve 18 controls a flow rate of a gas such as air, or a liquid such as a physiological saline solution, which flows inside a air/water supply tube (unillustrated). Also, based on the operations on the switch 86 of the operation section 34, the solenoid valve 18 controls a flow rate of a gas or a liquid suctioned through a suction tube (unillustrated).

The monitor 20 displays images observed by an observation optical system 40, described later, of the endoscope 12, and displays states of the endoscope 12.

The endoscope (endoscope main body) 12 includes an insertion section 32 to be inserted into a body cavity of a subject, an operation section 34 connected to a proximal end of the insertion section 32, a universal cable 36 extended from the operation section 34; and a connector (external-force input section, connection release means) 38 provided at a position of the universal cable 36 which is provided far from the operation section 34. Further, an illumination optical system and an observation optical system 40 are inserted through the insertion section 32, operation section 34, universal cable 36, and connector 38.

The illumination optical system includes an illumination lens 42 (see FIG. 1B), and an unillustrated light guide bundle provided in a proximal end side of the illumination lens 42. The observation optical system 40 includes an objective lens unit 44 (see FIG. 1B), a solid-state imaging element (unillustrated imaging unit), and an imaging cable (unillustrated). The objective lens unit 44 forms an image of light incident on the objective lens unit 44. The solid-state imaging element is provided in the proximal end side of the objective lens unit 44. Further, the solid-state imaging element is, for example, a CCD or CMOS, which picks up an image formed by the objective lens unit 44 and photoelectrically converts the image into an electric signal. The imaging cable transmits the electric signal photoelectrically converted by the solid-state imaging element to the connector 38 through the universal cable 36.

Various structures may be adopted for the observation optical system 40, such as a structure in which an ocular section is provided on the operation section 34 and the object can be observed through the ocular section (regardless of whether the monitor 20 is provided or not), or a structure which includes the objective lens unit 44 (see FIG. 1B), an image guide fiber, and an ocular section (regardless of whether the monitor 20 is provided or not).

As illustrated in FIG. 1A, the insertion section 32 includes a hard tip section 52, a bending section 54, and a flexible pipe section 56 which are provided in this order from a distal end side toward a proximal end side.

Distal ends of the illumination optical system, observation optical system 40, air/water supply tube, and suction tube are fixed to the hard tip section 52. Further, as illustrated in FIG. 1B, two illumination lenses 42 of the illumination optical system, the objective lens unit 44 of the observation optical system 40, a tip opening 62 in the suction tube, and an air/water supply nozzle 64 are provided on a distal end surface 52a of the hard tip section 52.

A tip end of the unillustrated light guide bundle is fixed to the hard tip section 52 behind the illumination lenses 42. The solid-state imaging element is fixed behind the objective lens unit 44.

Further, proximal ends of the air/water supply tube and suction tube are connected to the connector 38 through the bending section 54 and flexible pipe section 56 of the insertion section 32, the operation section 34, and the universal cable 36. Further, the suction tube is branched into a treatment-instrument insertion port 88a formed in a bending preventer 88, described later, of the operation section 34.

The bending section 54 is coupled to a bending operation knob 84, described later, of the operation section 34 by an unillustrated operation wire so that the bending section 54 is bent by operating the bending operation knob 84, and includes a bending pipe with plural bending pieces and an outer tube covering the exterior of the bending pipe.

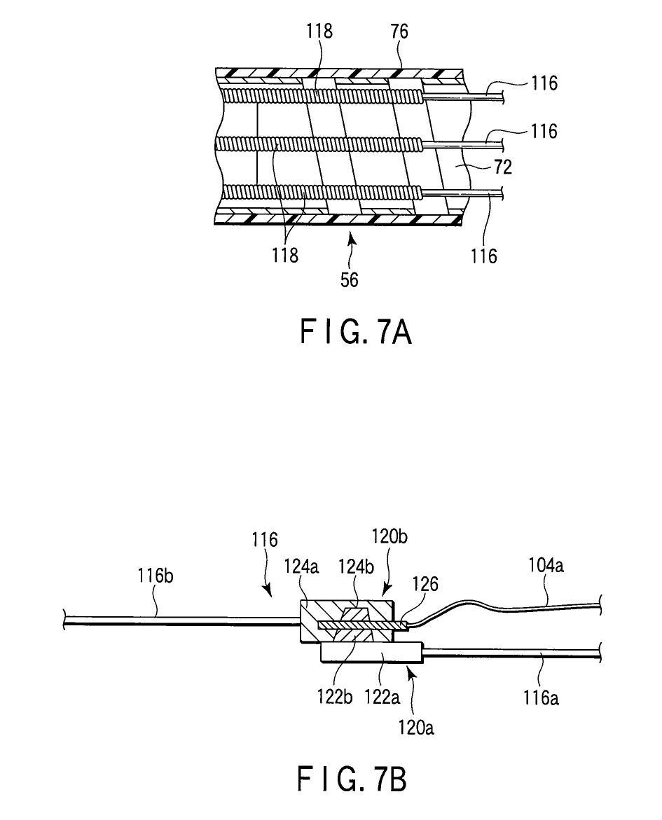

As illustrated in FIG. 3, the flexible pipe section 56 includes a helical tube 72, a braid 74 provided outside the helical tube 72, and an outer cover 76 provided outside the braid 74. The helical tube 72 is made of a thin strip of metal plate which is formed in a spiral shape. The braid 74 is made of a reticularly woven material shaped like a pipe. The outer cover 76 is made of a resin material formed in a tubular shape. The braid 74 need not always be provided (see FIG. 7A for the third embodiment described later).

The flexible pipe section 56 is formed to have appropriate flexibility. However, the flexible pipe section 56 used in this embodiment is more flexible (i.e., formed more bendy) than a flexible pipe section which is commonly used in endoscopes. A degree of flexibility can be adjusted by appropriately setting the flexibility of the helical tube 72, braid 74, and outer cover 76. Such a flexibility of the flexible pipe section 56 is determined in the balance with wires 104 described later. The flexibility of the flexible pipe section 56 is preferably such that does not allow the flexible pipe section 56, in an original state thereof, to be used as the flexible pipe section 56 for the endoscope 12. For example, the degree of flexibility of the flexible pipe section 56 preferably allows it to be easily bent into a free shape (so-called flaccid).

Further, in the flexible pipe section 56, four wire insertion holes 78 into which wires (detachment section, detachment means) 104 for adjusting the flexibility of the flexible pipe section 56 are respectively inserted, are fixed inside the helical tube 72. These wire insertion holes 78 are fixed to an inner circumferential surface of the helical tube 72 at intervals of substantially 90 degrees about the center axis of the helical tube 72. The shape of the wire insertion holes 78 can be appropriately set, and various shapes are allowable including; for example, a linear shape relative to the inner circumferential surface of the helical tube 72, and a spiral shape. FIG. 3 illustrates an example in which the wire insertion holes 78 are fixed to be linear relative to the inner circumferential surface.

As illustrated in FIG. 1A, the operation section 34 includes an operation section main body 82, a bending operation knob 84 including a stopper 84a, a switch 86, and a bending preventer 88. A treatment-instrument insertion port 88a coupled with the suction tube provided in the insertion section 32 is formed on the operation section body 82. Further, the endoscope treatment instrument α is inserted from the treatment insertion port 88a of the endoscope 12 into the suction tube, and is further pushed into the side of the hard tip section 52. The endoscope treatment instrument α is then protruded outside from a tip opening 62 of the suction tube.

Also, the operation section main body 82 is provided with the bending operation knob 84 and the switch 86. The bending operation knob 84 is operated when the bending section 54 of the insertion section 32 is to be bent. The switch 86 is operated when gas and/or water is supplied to the object, and blood and physiological saline solution near the object is suctioned. The switch 86 is provided with a signal line (unillustrated) inside the universal cable 36, to be electrically connected to the processor 16. A distal end of the signal line is provided at the electric connector 94, described later, of the connector 38.

The operation section main body 82 is also provided with the bending preventer 88 of the flexible pipe section 56. Further, the bending preventer 88, in cooperation with the operation section main body 82, constitutes a grip section which the user of the endoscope 12 grips.

The universal cable 36 is extended from the operation section main body 82. The universal cable 36 has a structure which is similar, for example, to that of the flexible pipe section 56 of the insertion section 32 illustrated in FIG. 3. Further, the universal cable 36 is formed to have an appropriate flexibility, like the flexible pipe section 56. The universal cable 36 used in this embodiment is more flexible (i.e., formed more bendy) than a universal cable which is commonly used in endoscopes. Therefore, the flexibility of the universal cable 36 preferably is of such a degree that does not allow the universal cable 36, in an original state thereof, to be used as the universal cable 36 for the endoscope 12. For example, at such a degree of flexibility, the universal cable 36 is allowed to be easily bent into a free shape (so-called flaccid).

Further, at a far end of the universal cable 36 relative to the operation section main body 82, the connector 38 is provided. As illustrated in FIG. 2A, the connector 38 includes a body 38a, a light guide connector (engaging section, engagement means) 92, an electric connector 94 provided at an end of a cable 94a, a gas/water supply cap 96, and a suction cap 98. The body 38a is formed in a substantially cylindrical shape or a substantially columnar shape.

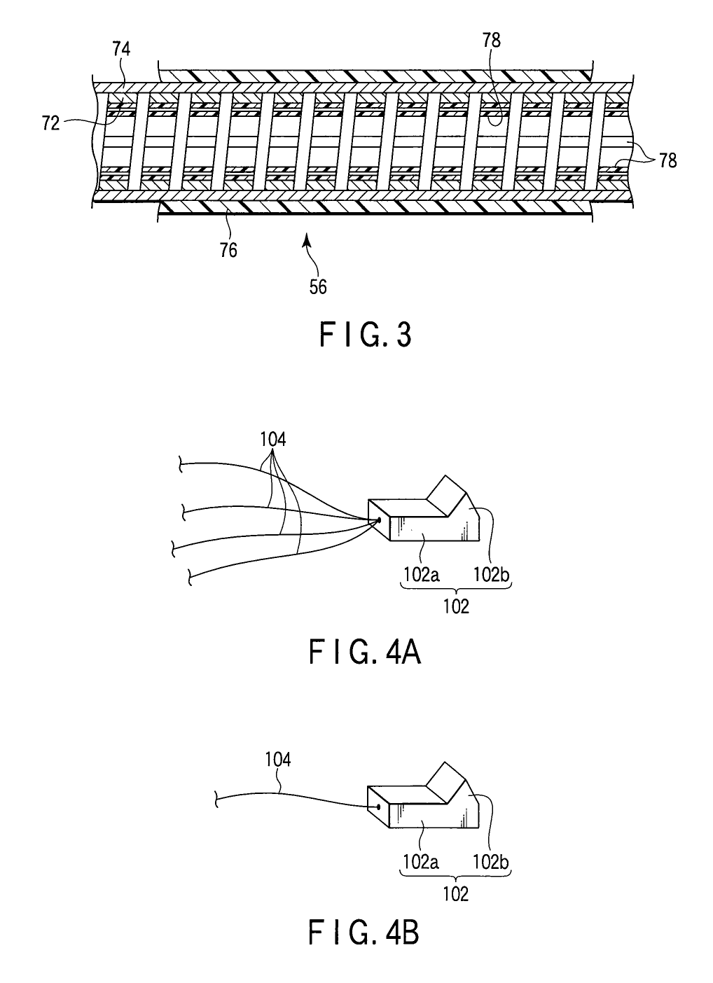

The light guide connector 92 is provided at a distal end of the body 38a of the connector 38 (in the side of the light source device 14 which is connected to the light-source-device-connector receiving section 22). As illustrated in FIG. 2A and FIG. 2B, the light guide connector 92 includes a light guide rod 92a and a concave 92b. The light guide rod 92a is provided to make illumination light enter the light guide bundle from the light source device 14. On the other hand, the claw member 102 (see FIG. 4A) connected to the wires 104, described later, is provided at the concave 92b.

The cable 94a is extended from a side of the body 38a of the connector 38. The electric connector 94 is provided at an end of the cable 94a. The electric connector 94 is detachably/attachably engaged with the processor-connector receiving section 24 of the processor 16.

Further, the air/water supply cap 96 and the suction cap 98 are provided in the side of the connector 38. A gas supply pump and a water supply pump, which are not illustrated, are detachably/attachably connected to the air/water supply cap 96. A suction pump and a suction pot, which are not illustrated, are detachably/attachably connected to the suction cap 98.

As illustrated in FIG. 2A and FIG. 4A, the claw member (engaging section, engagement means, retaining section, engagement retaining means) 102 disposed in the concave 92b of the light guide connector 92 is formed in an L-shape. The claw member 102 includes a substantially rectangular content section 102a contained in the concave 92b, and a claw section 102b extended sideward from the content section 102a. A tip end of the claw member 102 protrudes outward beyond the outer circumference of the light guide connector 92 when the claw member 102 is provided in the concave 92b. Therefore, the claw section 102b of the claw member 102 is engaged in a concave 22a of the light-source-device-connector receiving section 22 when the light guide connector 92 is connected to the light-source-device-connector receiving section 22 of the light source device 14.

Proximal ends of the wires 104 having the content section 102a of the claw member 102 having an appropriate stiffness are fixed to the content section 102a. The wires 104 are provided over the connector 38, universal cable 36, operation section 34, and insertion section 32. Further, distal ends of the wires 104 are provided to be freely movable inside the hard tip section 52 of the insertion section 32 and inside the bending section 54. The wires 104 give the universal cable 36 and the flexible pipe section 56 of the insertion section 32 an appropriate stiffness. That is, the wires 104 function to reinforce the flexible pipe section 56 and the universal cable 36. In particular, the flexible pipe section 56 and the universal cable 36 in this embodiment are much more flexible than a flexible pipe section and a universal cable which are commonly used. Therefore, when the wires 104 are not inserted, the flexibilities of the flexible pipe section 56 and universal cable 36 differ widely from a desired degree. On the other hand, when the wires 104 are inserted, the flexible pipe section 56 and universal cable 36 are adjusted to be less flexible due to stiffness of the wires 104, and attain flexibilities of a desired degree.

Various types of wires 104 are used, such as single wires or stranded wires made of a material such as carbon steel, stainless steel, glass fiber, or plastic.

Next, an operation of the endoscope system 10 according to the embodiment will be described.

When the endoscope 12 is used, the light guide connector 92 of the connector 38 is attached to the light-source-device-connector receiving section 22 of the light source device 14. Further, the electric connector 94 of the connector 38 is attached to the processor-connector receiving section 24 of the processor 16. Further, a gas supply pump and a water supply pump (both unillustrated) are connected to the gas/water supply cap 96. A suction pump and a suction bottle (both unillustrated) are attached to the suction cap 98. At this time, as illustrated in FIG. 2A, the claw member 102 is engaged in the concave 92b of the light-source-device-connector receiving section 22. Therefore, the wires 104 are inserted in the universal cable 36, operation section 34, and insertion section 32. Accordingly, the flexible pipe section 56 of the insertion section 32 of the endoscope 12, and the universal cable 36 each have such a degree of flexibility which allows easy use.

In this state, the insertion section 32 of the endoscope 12 is inserted into a body cavity. The endoscope treatment instrument α is inserted from the treatment-instrument insertion port 88a, and is protruded from the tip end opening 62. An appropriate treatment is carried out while observing an object.

After use of the endoscope 12, the light guide connector 92 of the connector 38 is detached from the light-source-device-connector receiving section 22 of the light source device 14. At this time, the light guide connector 92 of the connector 38 moves apart from light-source-device-connector receiving section 22 of the light source device 14. However, the claw member 102 fixed to the proximal ends of the wires 104 continues to be engaged in the concave 22a of the light-source-device-connector receiving section 22 of the light source device 14 (operates as a retaining section or an engagement retaining means which maintains engagement). Therefore, the claw member 102 is left in the concave 22a of the light-source-device-connector receiving section 22. On the other hand, the distal ends of the wires 104 are not fixed to the hard tip section 52 and the inside of the operation section 34 but are free. Therefore, the wires 104 fixed to the claw member 102 are gradually pulled out of the endoscope 12. That is, the distal ends of the wires 104 reach the flexible pipe section 56 from the hard tip section 52 of the insertion section 32 or the operation section 34.

Further, the distal ends of the wires 104 are pulled out up to the operation section 34 of the endoscope 12. Then, the flexible pipe section 56, which has been made stiff by inserting the wires 104 having stiffness through the flexible pipe section 56, gradually loses stiffness from the distal end side of the flexible pipe section 56 to the proximal end side thereof as the wires 104 are pulled out. Once the distal ends of the wires 104 are pulled out up to the operation section 34, the wires 104 cannot be inserted into the wire insertion holes 78 of the flexible pipe section 56 of the insertion section 32.

Therefore, the flexible pipe section 56 itself of the insertion section 32 of the endoscope 12 becomes so bendy that the flexible pipe section 56 can be easily folded. That is, if the hard tip section 52 is brought into contact with a body wall when the insertion section 32 is being inserted into a body cavity from the hard tip section 52, the flexible pipe section 56 easily bends, and the shape of inner conduits inside the flexible pipe section 56 cannot be maintained, depending on a degree of flexibility of the flexible pipe section 56.

Thus, the endoscope 12 comprising the insertion section 32, which has the too flexible pipe section 56, provides the insertion section 32 with poor operability for insertion into a body cavity, and transmits less torque when the insertion section 32 is pivoted about its axis. Therefore, the endoscope 12 can hardly be used even if the light guide connector 92 of the endoscope 12 is attached again to the connector receiving section 22 of the light source device 14 after the light source connector 92 of the connector 38 of the disposable type endoscope 12 is once attached to and detached from the light source device 14. Accordingly, reuse of the endoscope 12 can be effectively prevented.

As has been described above, the following can be said according to this embodiment.

The light guide connector 92 of the connector 38 of the endoscope 12 is once attached to the light-source-device-connector receiving section 22 of the light source device 14. Thereafter, the light guide connector 92 of the connector 38 of the endoscope 12 is detached from the light-source-device-connector receiving section 22 of the light source device 14. At this time, the wires 104 which make the flexible pipe section 56 of the insertion section 32 and the universal cable 36 stiff are pulled out from the endoscope 12. Therefore, in particular, the stiffness (flexibility) of the flexible pipe section 56 of the insertion section 32 can be made too low to be suitably used for the endoscope 12.

Further, once the distal ends of the wires 104 are pulled out from the insertion section 32 up to the operation section 34, the wires 104 cannot be inserted again into the wire insertion holes 78 of the flexible pipe section 56. Therefore, reuse of the disposable type endoscope 12 can be effectively prevented.

In this embodiment, it has been described that the concave 92b where one claw member 102 is provided is formed in the light guide connector 92, and the concave 22a to be engaged with the claw member 102 is formed in the light-source-device-connector receiving section 22 of the light source device 14. As an alternative, for example, two to four concaves 92b may be formed in the light guide connector 92, and the same number of concaves 22a as that of the concaves 92b may be formed in the light-source-device-connector receiving section 22. The claw member 102 illustrated in FIG. 4B may be provided in each of the concaves 92b and 22a. That is, one wire 104 may be fixed to the claw member 102, and distal ends of the wires 104 may be provided such that the distal ends of the wires 104 each reach the bending section 54 of the insertion section 32 of the endoscope 12.

Although the structure is configured to provide four wire insertion holes 78 in the inner circumferential surface of the helical tube 72 of the flexible pipe section 56, the number of wire insertion holes 78 is not limited to four. For example, two or three is also possible. That is, depending on the degree of flexibility of the wires 104, two or three wires 104 may be fixed to the claw member 102. If two wire insertion holes 78 are provided in the inner circumferential surface of the helical tube 72, the two wire insertion holes 78 are preferably provided at positions symmetrical to each other (at opposed positions). Alternatively, if three wire insertion holes 78 are provided, the wire insertion holes 78 are preferably provided, for example, at intervals of 120 degrees about the center axis of the helical tube 72. Alternatively, the number of wire insertion holes 78 may be appropriately set depending on the stiffness of the wires 104.

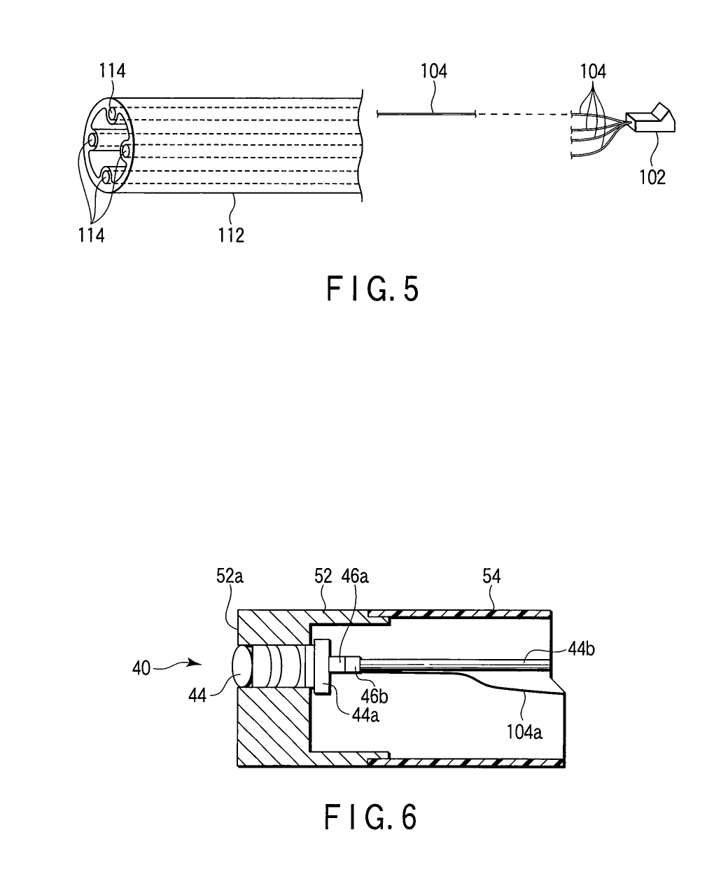

Also in this embodiment, it has been described that the flexible pipe section 56 of the insertion section 32 of the endoscope 12 and the universal cable 36 includes the helical tube 72 (braid 74) and the outer cover 76, as illustrated in FIG. 3. Alternatively, in place of such a flexible pipe section 56 and universal cable 36, a tube 112 in which the wire insertion holes 114 are formed may be used, as illustrated in FIG. 5. The tube 112 needs to be flexible (bendy) and is not particularly limited. However, the tube 112 may be formed of, for example, a plastic material such as polyethylene (PE), polypropylene (PP), polyvinyl alcohol (PVA), or a layered material thereof.

Further alternatively, the structure of the concave 92b of the light guide connector 92 of the endoscope 12 may be employed in the electric connector 94 of the light source device 14, and the structure of the concave 22a in the connector receiving section 22 of the light source device 14 may be employed in the processor-connector receiving section 24 of the processor 16, preferably. Further, the claw member 102 may be provided on the electric connector 94, and the wires 104 may be provided in the electric connector 94, cable 94a, universal cable 36, operation section 34, and insertion section 32.

### Second Embodiment

Next, the second embodiment will be described with reference to FIG. 6. This embodiment is a modification of the first embodiment. The same members or members having the same operations as in the first embodiment will be denoted with the same reference symbols as in the first embodiment, and detailed descriptions thereof will be omitted herefrom.

As illustrated in FIG. 6, an observation optical system 40 includes an objective lens unit 44 (see FIG. 1B), a solid-state imaging element (imaging unit) 44a, and an imaging cable 44b. Of these members, an outer circumferential surface of the objective lens unit 44 in a distal end side thereof is provided in a hard tip section 52. A proximal end of the objective lens unit 44 is fixed to the solid-state imaging element 44a. Further, an imaging-element-side connector (connection section) 46a is formed on a surface of the solid-state imaging element 44a in a side opposite to a surface where the objective lens unit 44 is provided. An imaging-cable-side connector (connection section) 46b which is detachable from the imaging-element-side connector 46a is formed on a distal end of the imaging cable 44b. That is, detachable connectors (connection section) 46a and 46b are provided between the solid-state imaging element 44a and the imaging cable 44b.

Distal ends of wires (detachment section, detachment means) 104a which substitute for wires 104 described in the first embodiment are fixed (connected) to the imaging-cable-side connector 46b. Connection between the imaging-element-side connector 46a and the imaging-cable-side connector 46b is adjusted as follows. When the wires 104a are strongly pulled out with the imaging-element-side connector 46a fixed, for example, to a certain part, the connection between the imaging-element-side connector 46a and the imaging-cable-side connector 46b is released without cutting the wires 104a.

Proximal ends of the wires 104a are fixed to a content section 102a of a claw member 102, like wires 104 described in the first embodiment. That is, the wires 104a are provided inside the endoscope 12, like the wires 104 described in the first embodiment. Further, the wires 104a may be provided to adjust the flexibility of a flexible pipe section 56 and a universal cable 36, as described in the first embodiment (may have an adjustment ability to adjust flexibility). Alternatively, the wires 104a may have no adjustment ability as described but may be simply used to connect the claw member 102 to an imaging-cable-side connector 46b of the observation optical system 40. Since the former wires have been described in the first embodiment, this embodiment will be described as comprising the latter wires.

Next, an operation of an endoscope system 10 according to this embodiment will be described.

With a connector 38 of an endoscope 12 connected to a connector receiving section 22 of a light source device 14, an insertion section 32 of the endoscope 12 is inserted into a body cavity. An endoscope treatment instrument α is inserted from a treatment-instrument insertion port 88a, and is protruded from a tip end opening 62. An appropriate treatment is carried out while observing an object.

After use of the endoscope 12, a light guide connector 92 of the connector 38 is detached from the light-source-device-connector receiving section 22 of the light source device 14. At this time, the light guide connector 92 of the connector 38 moves apart from the light-source-device-connector receiving section 22 of the light source device 14. However, the claw member 102 fixed to the proximal ends of the wires 104 continues to be engaged in the concave 22a of the light-source-device-connector receiving section 22 of the light source device 14 (maintains engagement). Therefore, the claw member 102 is left in the concave 22a of the light-source-device-connector receiving section 22.

On the other hand, distal ends of the wires 104 are fixed to the imaging-cable-side connector 46b, and an imaging-element-side connector 46a is formed on the solid-state imaging element 44a to which an objective lens unit 44 provided at a hard tip section 52 is fixed. That is, the solid-state imaging element 44a is fixed near a distal end of the insertion section 32 of the insertion section 12. Therefore, the wires 104a are more tensioned as the connector 38 of the endoscope 12 is moved more apart from the connector receiving section 22 of the light source device 14. That is, a force of releasing connection between the imaging-element-side connector 46a and the imaging-cable-side connector 46b is applied. Accordingly, the connection between the imaging-element-side connector 46a and the imaging-cable-side connector 46b is finally released.

Further, the imaging-element-side connector 46a and the imaging-cable-side connector 46b, connection between which has been once released, stay inside the endoscope 12. Therefore, the imaging-element-side connector 46a and the imaging-cable-side connector 46b cannot be connected to each other without disassembling the endoscope 12.

Therefore, the solid-state imaging element 44a in the observation optical system 40 can neither be supplied with electric power nor transmit/receive data of images picked up by the solid-state imaging element 44 any more. That is, the observation optical system 40 of the endoscope 12 cannot be used any more, and the observation function of the endoscope 12 can therefore not be used any more. Accordingly, reuse of the endoscope 12 can be prevented effectively.

Although this embodiment has described the objective lens unit 44 as being fixed to the solid-state imaging element 44a, the objective lens unit 44 and the solid-state imaging element 44a may be detachable or the imaging cable 44b may be separated into two halves in axial directions. That is, in this embodiment, it has been described that the imaging-element-side connector 46a is formed on the solid-state imaging element 44a, and the imaging-cable-side connector 46b is formed on the imaging cable 44b. The connectors 46a and 46b may also be provided on the imaging cable 44b. Therefore, the connectors 46a and 46b need not always be provided inside the insertion section 32, and may be provided inside the operation section 34, universal cable 36, or connector 38. In this case, the wires 104a may be shorter than when the wires 104a are provided inside the insertion section 32. Further, the connectors 46a and 46b may be provided on a signal line and/or a power line (both unillustrated) provided in the imaging cable 44b. That is, the connectors 46a and 46b may be provided at any position on the imaging cable 44b.

### Third Embodiment

Next, the third embodiment will be described with reference to FIG. 7A and FIG. 7B. This embodiment is a modification of the first and second embodiments. The same members or members having the same operations as in the first and second embodiments will be denoted by the same reference symbols as in the first and second embodiments, and detailed descriptions thereof will be omitted herefrom.

To bend a bending section 54 of an insertion section 32 of an endoscope 12, four operation wires 116 are provided through angle coils 118 inside a flexible pipe section 56, as illustrated in FIG. 7A. Distal ends of the angle coils 118 are fixed to, for example, an inner circumferential surface of the closest bending piece to a proximal end side of an unillustrated bending pipe in the bending section 54.

For example, as illustrated in FIG. 7B, first and second connection sections 120a and 120b which connect the operation wires 116 are provided on at least one of the four operation wires 116. These connection sections 120a and 120b connect an operation-section-side wire 116a proximal to an operation section 34 and a distal-end-side operation wire 116b proximal to a distal end of the insertion section 32.

The first connection section 120a comprising a base 122a and a convex 122b is provided at a distal end of the operation-section-side operation wire 116a (in a near side to the distal end of the insertion section 32). The base 122a thereof is fixed to a distal end of the operation-section-side operation wire 116a. The convex 122b is formed integrally on the base 122a.

The second connection section 120b comprising a base 124a and a concave 124b is provided at a proximal end of the distal-end-side operation wire 116b (in a near side to the operation section 34). The base 124a thereof is fixed to a proximal end of the distal-end-side operation wire 116b. The concave 124b is formed integrally in the base 124a, and is formed so as to receive the convex 122b of the first connection section 120a.

A cross section of a part of the first connection section 120a that is engaged in the concave 124b of the second connection section 120b is formed in a substantially trapezoidal shape (a substantially truncated conical shape as a whole). Further, axial directions (center axes) of the convex 122b of the first connection section 120a and the concave 124b of the second connection section 120b are perpendicular to axial directions of the operation wires 116. Therefore, even when the convex 122b of the first connection section 120a is engaged in the concave 124b of the second connection section 120b, the first connection section 120a easily falls out.

Further, a pin 126 is inserted in an appropriate direction such as a parallel or substantially parallel direction relative to, for example, the axial directions of the operation wires 116, with the convex 122b of the first connection section 120a inserted in the concave 124b of the second connection section 120b. The convex 122b of the first connection section 120a and the concave 124b of the second connection section 120b are thereby engaged with each other, with the pin 126 penetrated therethrough. The pin 126 is formed in a wedge shape. The pin 126 is inserted so as to penetrate the first connection section 120a and the second connection section 120b, with both sections engaged with each other. At this time, the wedge shape functions as a retainer for preventing the first connection section 120a from falling out of the second connection section 120b. The distal end of the wire 104a described in second embodiment is connected to the pin 126. On the other hand, the proximal end of the wire 104a is fixed to the content section 102a of the claw member 102.

Other features of the structure are the same as those described in the second embodiment, and detailed descriptions thereof will be omitted herefrom.

With the connector 38 of the endoscope 12 connected to a connector receiving section 22 of a light source device 14, the insertion section 32 of the endoscope 12 is inserted into a body cavity. An endoscope treatment instrument α is inserted from a treatment-instrument insertion port 88a, and is protruded from a tip end opening 62. An appropriate treatment is carried out while observing an object.

After use of the endoscope 12, a light guide connector 92 of the connector 38 is detached from the light-source-device-connector receiving section 22 of the light source device 14. At this time, the light guide connector 92 of the connector 38 moves apart from the light-source-device-connector receiving section 22 of the light source device 14. However, the claw member 102 fixed to the proximal end of the wire 104a continues to be engaged in the concave 22a of the light-source-device-connector receiving section 22 of the light source device 14 (maintains engagement). Therefore, the claw member 102 is left in the concave 22a of the light-source-device-connector receiving section 22.

On the other hand, the distal end of the wire 104a is fixed to the proximal end of the pin 126 penetrating through the convex 122b of the first connection section 120a and the concave 124b of the second connection section 120b, the connection sections 120a and 120b connecting the operation-section-side operation wire 116a and the distal-end-side operation wire 116b of the insertion section 32. Therefore, the wire 104a is more tensioned as the connector 38 of the endoscope 12 is moved more apart from the connector receiving section 22 of the light source device 14. That is, the pin 126 is applied with a force of pulling off the pin 126 from a state of penetrating through the convex 122b of the first connection section 120a and the concave 124b of the second connection section 120b.

Finally, the pin 126 is pulled out, from the state of penetrating through the convex 122b of the first connection section 120a and the concave 124b of the second connection section 120b. At this time, a tension, which was applied when the endoscope 12 was manufactured, and a further tension or pressure, which is applied to the operation wires 116 by operating the operation section 34 to move the operation wires 116 forward and backward along axial directions thereof, are transmitted to the concave 124b and base 124a of the second connection section 120b through the base 122a and concave 122b of the first connection section 120a. At this time, the convex 122b having the substantially truncated conical shape of the first connection section 120a receives a force from a slope of the concave 124b having the substantially truncated conical shape of the second connection section 120b, in a direction deviating from axial directions of the operation wires 116. Accordingly, the convex 122b having the substantially truncated conical shape of the first connection section 120a slides along the slope of the concave 124b having the substantially truncated conical shape of the second connection section 120b, thereby to release connection between the first and second connection sections 120a and 120b.

On the other hand, the convex 122b of the first connection section 120a and the concave 124b of the second connection section 120b, connection between which has once been released, stay inside the endoscope 12 and can therefore not be connected without disassembling the endoscope 12, nor can the pin 126 be positioned so as to penetrate through the convex 122b of the first connection section 120a and the concave 124b of the second connection section 120b any more.

Then, at least one operation wire 116 among the four operation wires 116 is put in the same state as if the at least one operation wire 116 was cut. Therefore, an operation force for operating the operation wires 116 cannot properly be transmitted to the bending section 54 through the operation wires 116. Accordingly, the bending section 54 cannot be properly bent. That is, bending operations cannot be properly carried out, and the bending function of the endoscope 12 cannot be utilized. Accordingly, reuse of the endoscope 12 can be effectively prevented.

The first and second connection sections 120a and 120b may be provided at any positions insofar as the positions are within a part where the operation wires 116 are provided, between the operation section 34 and the distal end side of the insertion section 32. However, from a viewpoint of preventing increase in diameter of the insertion section 32, the first and second connection sections 120a and 120b are preferably provided at positions inside the operation section 34, or at near positions to the operation section 34 inside the insertion section 32.

### Fourth Embodiment

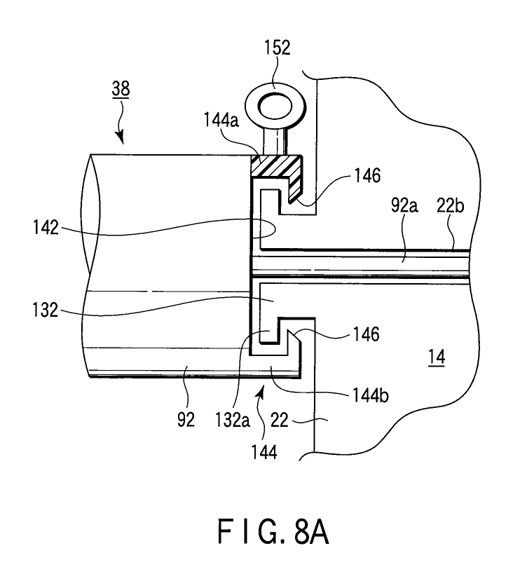

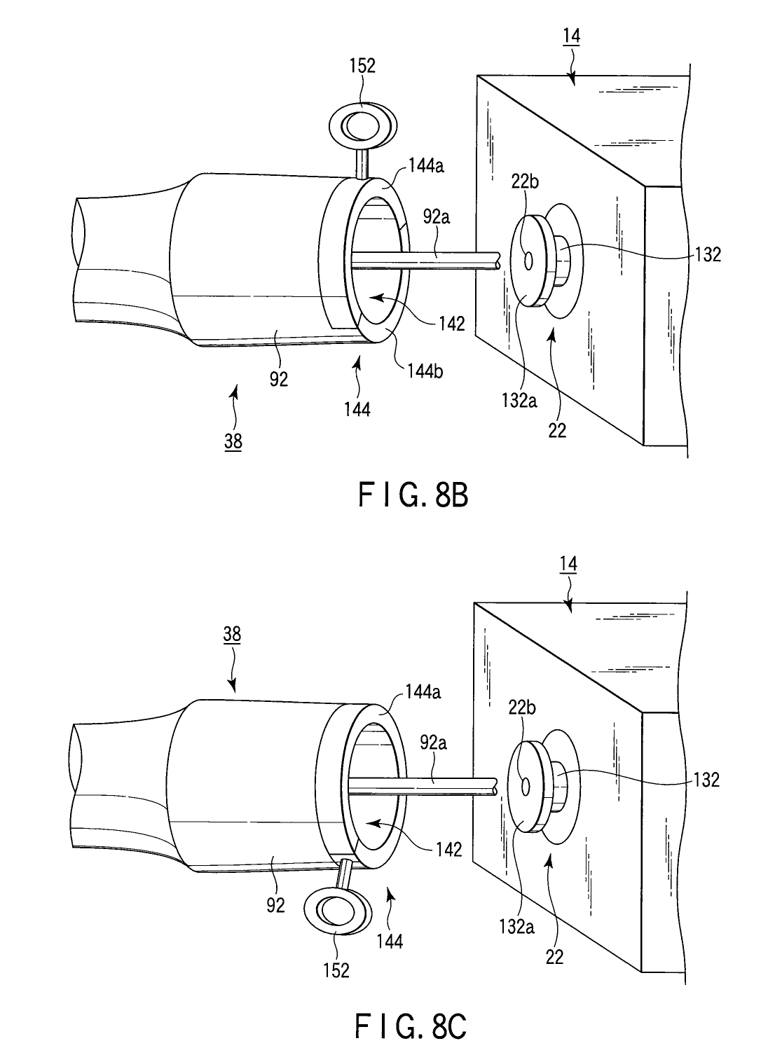

Next, the fourth embodiment will be described with reference to FIG. 8A to FIG. 8C. This embodiment is a modification to the first embodiment. The same members or members having the same operations as in the first embodiment will be denoted by the same reference symbols as in the first embodiment, and detailed descriptions thereof will be omitted herefrom.

As illustrated in FIG. 8A and FIG. 8B, a connector receiving section 22 in a light source device 14 according to this embodiment differs from the connector receiving section 22 in the light source device 14 described in the first embodiment, and includes a convex 132 including a flange section (engaged section) 132a. Specifically, the convex 132 and flange section 132a form the connector receiving section 22 protruded outside the light source device 14.

On the other hand, a concave 142 having a substantially disc-like shape is formed on a distal end of a light guide connector 92 of an endoscope 12 (which is an end in a side connected to the connector receiving section 22 of the light source device 14).

Further, a concave 92b (see FIG. 2A and FIG. 2B) of the light guide connector 92 as described in the first embodiment is not formed in the light guide connector 92, and nether a claw member 102 nor wires 104 are provided.

Therefore, as illustrated in FIG. 8A and FIG. 8B, a toric engaging section 144 is formed at the distal end of the light guide connector 92 by the substantially disc-shaped concave 142. The engaging section 144 includes a slope 146 which is inclined from outside of the light guide connector 92 to inside thereof. An inclination direction of the slope 146 is high toward the outside and low toward the inside. Further, of the engaging section 144 of the concave 142, a region which slightly exceeds, for example, 180 degrees is made of a resin material, and remaining parts (regions) are made of a hard material (e.g., stainless steel). That is, the engaging section 144 includes a flexible section (engaging section, engagement means, detachment section, and detachment means) 144a, and a rigid section 144b.

When the light guide connector 92 of the endoscope 12 is provided at the connector receiving section 22 of the light source device 14, the slope 146 of the light guide connector 92 is brought into contact with the flange section 132a of the connector receiving section 22 of the light source device 14. Then, the flexible section 144a of the engaging section 144 of the concave 142 of the light guide connector 92 is elastically deformed, thereby overhanging the flange section 132a of the connector receiving section 22 of the light source device 14. Therefore, the light guide connector 92 of the endoscope 12 and the concave 132 of the connector receiving section 22 of the light source device 14 are engaged with each other.

Further, a finger hook section (external-force input section, connection release means, detachment section, detachment means) 152 is formed outside the flexible section 144a of the concave 142. The finger hook section 152 is formed in a ring-like shape to allow a finger to be hooked on, and is provided to separate the flexible section 144a from the rigid section 144b.

FIG. 8B illustrates an example in which the finger hook section 152 is provided near the center of the flexible section 144a. However, the finger hook section 152 may be provided at a position more proximal to an end because the flexible section 144a can then be more easily separated from the rigid section 144b with a weaker force.

As described above, the following can be said according to this embodiment.

When the light guide connector 92 of the endoscope 12 is detached from the convex 132 of the connector receiving section 22 of the light source device 14, the flexible section 144a of the engaging section 144 of the light guide connector 92 needs to be broken in relation to the rigid section 144b. Therefore, even if an attempt is made to attach the light guide connector 92 again to the convex 132 of the connector receiving section 22, the flexible section 144a, which occupies more than half of the engaging section 144, has been already broken. Therefore, stability is insufficient even if the light guide connector 92 can be attached. Accordingly, reuse of the endoscope 12 can be effectively prevented after the light guide connector 92 of the endoscope 12 of a disposable type is once attached to and then detached from the connector receiving section 22 of the light source device 14.

In this embodiment, the region of the engaging section 144 of the light guide connector 92 where the flexible section is provided has been described as exceeding 180 degrees (half of a circumference). However, as illustrated in FIG. 8C, the engaging section 144 may be constituted, for the whole circumference thereof, by the flexible section 144a (i.e., no rigid section 144b as illustrated in FIG. 8B may be provided).

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical device comprising:
a first medical device;
a second medical device attachable to/detachable from the first medical device;
an engagement mechanism including an engaging section provided on the first medical device, and an engaged section provided on the second medical device and engaged in the engaging section;
a connection release mechanism including an external-force input section applied with an external force for releasing connection between the first and second medical devices; and
a detachment mechanism including a detachment section where the engaging section or the engaged section is irreversibly detached from the second medical device when the external force is applied to the external-force input section.

2. An endoscope comprising:
an endoscope main body including an insertion section;
a wire which is inserted into the endoscope main body, is able to provide predetermined stiffness for the insertion section, and is able to be pulled out of the endoscope main body;
an engagement mechanism provided at a proximal end of the wire and including an engaging section which is able to be engaged in a connector receiving section attachable to/detachable from the endoscope main body; and
an engagement retaining mechanism provided on the engagement mechanism and including a maintenance section which maintains engagement with the connector receiving section after connection between the endoscope main body and the connector receiving section is released.

3. An endoscope comprising:
an endoscope main body including an insertion section;
a wire which is inserted into the endoscope main body and is able to be pulled out of the endoscope body;
an engagement mechanism provided at a proximal end of the wire and including an engaging section which is able to be engaged in a connector receiving section attachable to/detachable from the endoscope main body;
an engagement retaining mechanism provided on the engagement mechanism and including a retaining section which maintains engagement with the connector receiving section after connection between the endoscope main body and the connector receiving section is released; and
a connection section which is connected to a distal end of the wire and is located inside the endoscope main body after the connection between the endoscope main body and the connector receiving section is released, the connection section being separable to enable the endoscope body to function when the endoscope main body and the connector receiving section are connected to each other or to prevent the endoscope main body from functioning when the endoscope body and the connector receiving section are separated from each other.

4. An endoscope comprising:
an endoscope main body including an insertion section;
an engagement mechanism provided on the endoscope main body and including an engaging section which is able to be engaged in a connector receiving section attachable to/detachable from the endoscope main body; and
a detachment mechanism provided on the engagement mechanism and including a detachment section which makes the engaging section be irreversibly detached from the endoscope main body when connection between the endoscope main body and the connector receiving section is released.
